(19) United States
(12) Patent Application Publication
(10) Pub. No.: US 2005/0055002 A1
(43) Pub. Date: Mar. 10, 2005

(54) Disposable sanitary undergarment

(75) Inventors: Welicia Whitelaw, Markham, IL (US); Tanya C. Smith, Chicago, IL (US)

Correspondence Address: WELSH & KATZ, LTD, 120 S RIVERSIDE PLAZA, 22ND FLOOR, CHICAGO, IL, 60606, US

(73) Assignee: Welicia Whitelaw, Markham, IL (US)

(21) Appl. No.: 10/654,714

(22) Filed: Sep. 4, 2003

(51) Int. Cl. A61F 13/15; A61F 13/20

(52) U.S. Cl. 604/385.03

(57) ABSTRACT

A disposable sanitary undergarment includes inside, outside, front, back, side, and crotch portions, as well as waist and leg openings. Resiliently elastic bands extend around outer peripheries of the waist and leg openings to facilitate holding the undergarment on a wearer. An absorbent pad is attached to the crotch portion of the undergarment. The absorbent pad has inwardly curved opposing longitudinal edges and arcuate lateral edges. An optional permeable cover portion may be attached to both sides of the crotch portion, enclosing the absorbent pad between the cover portion and the crotch portion. The optional cover portion follows the contour of the pad and also inwardly curved opposing longitudinal edges.

### BACKGROUND OF THE INVENTION

The present invention is directed to a disposable sanitary undergarment. More particularly, the present invention pertains to a disposable sanitary undergarment for use by both men and women as a sanitary napkin or an incontinence guard.

Regular undergarments in current use are made of cotton and/or synthetic materials. Such cotton and synthetic undergarments typically do not offer barrier protection. Often, synthetic undergarments have a cotton-lined crotch portion to absorb discharges or perspiration. The absorbent/barrier properties of regular undergarments are minimal such that any discharge and/or heavy perspiration may flow through onto outer clothing.

Sanitary napkins or incontinence pads used with regular undergarments generally have polyethylene backings that provide barrier properties needed to prevent liquid flow through. However, if discharge extends to the sides or the ends of the pads, it can leak onto the undergarment. This leakage can stain the undergarment. Depending upon the amount of leakage, liquid may flow through or go around the undergarment to stain outer clothing and/or bedding as well.

A majority of sanitary or incontinence pad users experience at least some leakage of fluids onto their undergarments. Normally, this leakage occurs at the side of the product, although end leakage also is a problem. Placement of absorbent pads in the crotch portion of regular undergarments shows that, at best, pads lay on undergarment leg elastic and, at worst, overhang the leg elastics, causing additional leakage. Typical leakage is caused by a poor fit of the pad to the body, improper positioning of the pad by the wearer, and lack of absorbency. Leakage from the undergarment onto the outer clothing is due to incompatibility between pad width and undergarment crotch width, and lack of barrier properties in the undergarment material around the edge portion of a pad.

The use of disposable sanitary undergarments combined with sanitary napkins or incontinence pads is well known in the art, including U.S. Pat. Nos. 6,428,524; 6,367,089; 6,231,558; 5,441,493; 4,560,381; 3,420,236; 3,232,293; 2,748,772; and 2,102,359. These undergarments generally are made of low-cost materials, such as paper, cellulose, or unwoven fabric. Many of these undergarments include absorbent pads and/or barrier means, to protect wearers from discharges coming into contact with their outer clothing. Unfortunately, disposable undergarments made with inexpensive materials such as paper or cellulose tend to appear very unattractive and unrefined. The necessary relative bulkiness of these undergarments, together with their unappealing appearance, make them resemble children's diapers, rendering potential wearers reluctant to purchase them.

Accordingly, there exists a need for a disposable sanitary undergarment, for both men and women, that is attractive in design and offers the wearer superior levels of security and convenience. Desirably, the undergarment is made using commercially available undergarments and sanitary/incontinence pads so that a cost-efficient product may be produced.

### BRIEF SUMMARY OF THE INVENTION

A disposable sanitary undergarment includes inside, outside, front, back, side, and crotch portions, as well as waist and leg openings. Resiliently elastic bands extend around outer peripheries of the waist and leg openings, holding the undergarment on a wearer. An absorbent pad is attached to the crotch portion of the undergarment. The absorbent pad has inwardly curved longitudinal sides and arcuate opposing lateral ends. A permeable cover portion may be attached to both sides of the crotch portion, enclosing the absorbent pad between the cover portion and the crotch portion. The cover portion has convex front and back arcuate ends, and a pair of convex sides.

In a preferred embodiment, the absorbent pad acts as a sanitary napkin. The absorbent pad may also, however, act as an incontinence pad or guard. The absorbent pad may be attached to the inside crotch portion of the undergarment by a variety of means, such as stitching, gluing, bonding, or heat treating. The cover portion may be attached by the same variety of means to the sides of the crotch portion.

In one embodiment, the undergarment is formed as a single integrated unit. In another embodiment, a side portion of the undergarment has a separation line extending between the waist opening and the adjacent leg opening. This separation line allows the undergarment to be separable along the separation line for easy removal from the wearer without requiring the wearer to slip his or her leg through the undergarment's leg opening. Such one-leg access allows the wearer access to the interior of the undergarment without requiring removal of the entire undergarment. Most preferably, complementary strips of Velcro are attached to either side of the separation line.

In a different embodiment, both side portions of the undergarment have separation lines extending between the waist opening and each adjacent leg opening. This separation line allows wearers to remove the entire garment without having to slip either leg through either of the undergarment's leg openings. Preferably, complementary strips of hook and loop fastening material are attached to both sides of the separation lines or portions.

Preferably, the undergarment is fashioned from a regular, commercially available undergarment made of cotton and/or synthetic materials, such as one might purchase at any clothing store. Similarly, the absorbent pad is a commercially available “off-the-shelf” absorbent pad, such as are available at drug stores, grocery stores and/or pharmacies. Preferably, the entirety of the absorbent pad is used when constructing the undergarment, although alternately, only a portion of the pad may be included.

Further objects, features, and advantages of the present invention, together with the organization and manner of use thereof, will become apparent from the following description of the invention when taken in conjunction with the accompanying drawings, wherein like reference numerals designate like elements throughout the several views.

While the present invention is susceptible to various modifications and alternative forms, specific embodiments thereof are shown by way of example in the accompanying drawings and will be described in detail. It should be understood that the drawings and detailed description thereof are not intended to limit the invention to the particular form disclosed, but rather the invention is intended to cover all modifications, equivalents, and alternatives falling within the spirit and scope of the present invention.

### BRIEF DESCRIPTION OF THE DRAWINGS

FIG. 1 is a front perspective view of a disposable sanitary undergarment in accordance with a specific embodiment of the present invention;

FIG. 2 is a side perspective view of the undergarment of FIG. 1;

FIG. 3 is a front perspective front view of an alternate embodiment of the present invention;

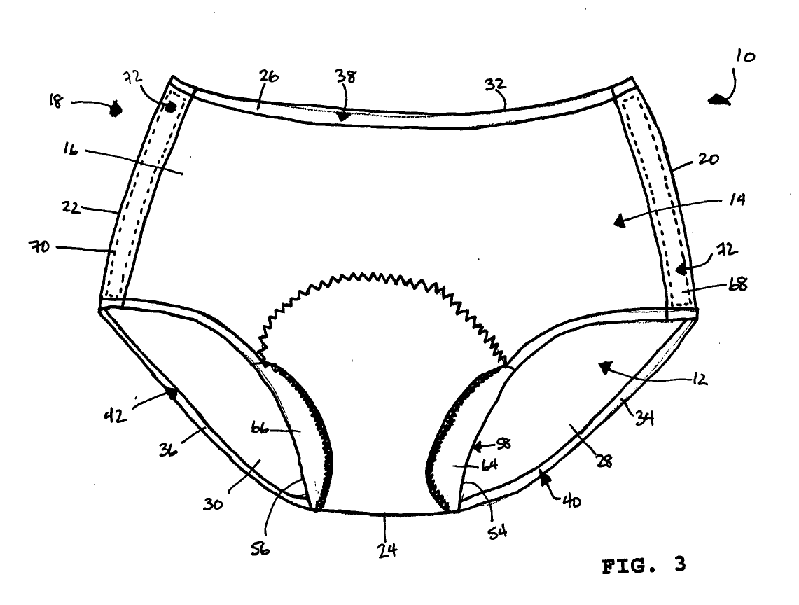

FIG. 4 is a side perspective view of the undergarment of FIG. 3;

FIG. 5 is a top plan view of the inside of a disposable sanitary undergarment separated along side separation lines of the undergarment; and

FIG. 6 is cross-sectional view of the present invention taken along line 6-6 of FIG. 5.

### DETAILED DESCRIPTION OF THE INVENTION

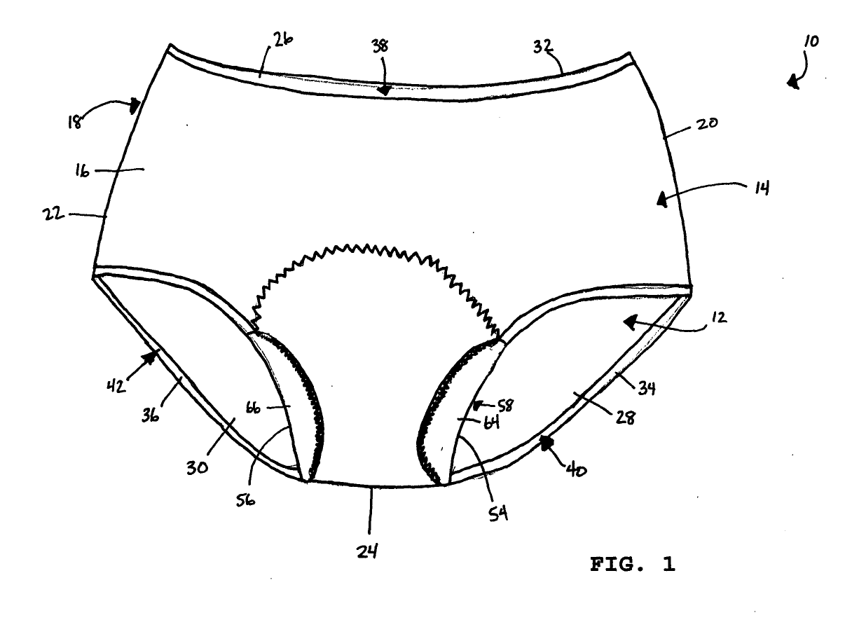

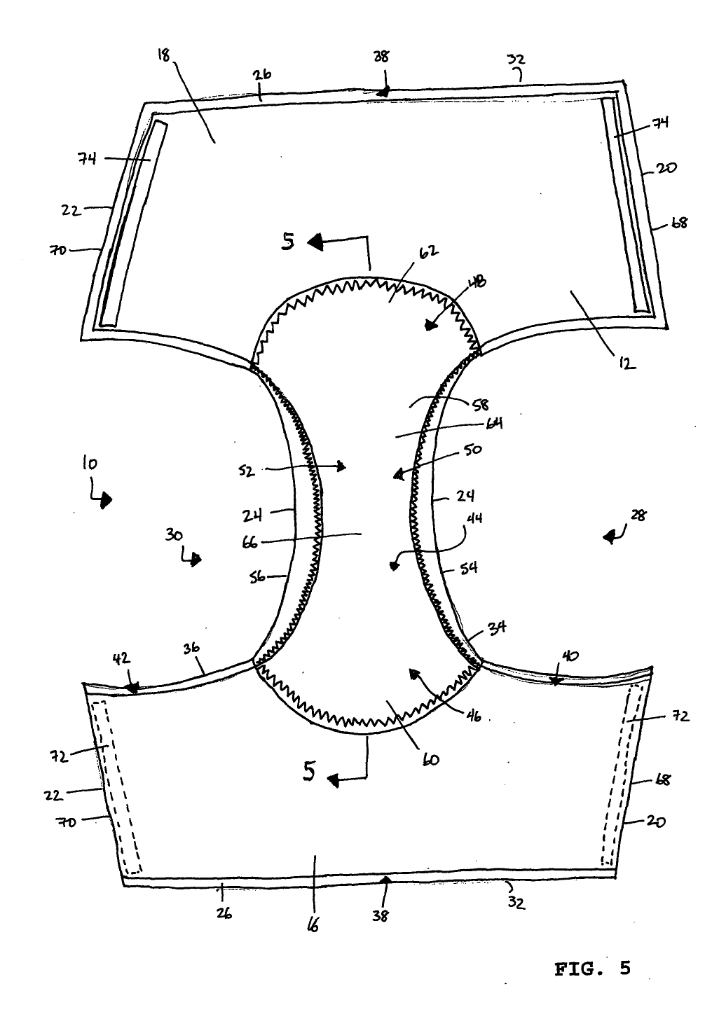

Referring now to FIGS. 1, 2, and 5, a disposable sanitary undergarment 10 is shown in accordance with the principles of the present invention. The undergarment 10 has an inside portion 12, an outside portion 14, front portion 16, back portion 18, first side portion 20, second side portion 22, a crotch portion 24, a waist opening 26, a first leg opening 28, and a second leg opening 30. The crotch portion 24 is located between the leg openings 28, 30.

The waist opening 26 has an outer peripheral edge 32 that circumscribes the waist of the wearer when worn. Each of the leg openings 28, 30 are defined by outer peripheral edges 34, 36. The undergarment 10 may further include a resiliently elastic waist band 38 extending around the outer periphery 32 of the waist opening 26 configured to hold the undergarment on the waist of a wearer, as is known in the art. Resilient elastic leg bands 40, 42 also extend around the peripheries 34, 36 of both of the leg openings 28, 30 and are designed to stretch as the legs of the wearer fully enter the leg openings. Thus, a snug fit is achieved to prevent leakage of fluids between the leg openings and the legs of the wearer.

A bottom surface 43 of an absorbent pad 44 is attached to the inside 12 of the crotch portion 24. The pad 44 may be configured to act either as a sanitary napkin or as an incontinence guard. Preferably, the pad 44 has arcuate front 46 and back 48 lateral sides and inwardly curved opposite longitudinal sides 50, 52. Thus, the shape appears somewhat like an “hourglass.” The absorbent pad is sufficiently long so that the front 46 and back 48 lateral sides 44 extend through the crotch portion 24 to attach to the front 16 and back 18 portions of the undergarment 10. The lateral sides 50, 52 of the pad 44 rest approximately along first 54 and second 56 sides of the crotch portion 24.

The first side 50 of the pad 44 lies adjacent the first leg opening 28, and the second side 52 of the pad lies adjacent the other, second leg opening 30. In one specific embodiment, the bottom surface 43 of the pad 44 may be attached to the inside 12 of the crotch portion 24 and to the undergarment's front 16 and back 18 portions by a variety of methods, including stitching, chemical adhesive, bonding, or heat treating. In FIGS. 1, 2, and 5, the bottom surface 43 of the pad 44 is represented as being attached to the crotch 24, front 16, and back 18 by stitching.

The pad 44 may be overlaid and enclosed by an optional cover portion 58, which attaches to the crotch portion 24 and completely encloses the pad between the crotch portion and the cover portion. The cover portion 58 may also extend around the crotch portion to the outside of the crotch portion so as to form a “wrap” around the entire crotch portion with the pad 44 enclosed therein.

The optional cover portion 58 is fluid-permeable to allow bodily fluids to pass through the cover portion into the absorbent pad 44. Preferably, the optional cover portion 58 follows the contour of the pad 44, thus the cover portion may also have inwardly curved opposite longitudinal sides 64, 66 and arcuate lateral sides 60, 62. Similarly, the opposite lateral sides 60, 62 of the optional cover portion 58 extend through the crotch portion 24 to attach to the front 16 and back 18 ends of the undergarment 10, and thus overlap and extend past the boundaries of the pad 44 that it encloses. The longitudinal sides 64, 66 of the cover 58 further extend past the inside edges 50, 52 of the crotch portion 24 to wrap around to attach to the outside 14 sides of the crotch portion.

The first side 64 of the optional cover portion 58 lies adjacent the first leg opening 28, and the second side 66 of the cover 58 lies adjacent the other, second leg opening 30. Thus, the optional cover portion 58 may span the width of the crotch portion, but need not necessarily do so. The cover 58 may be attached and/or affixed to the crotch portion 24 by a variety of methods, including stitching, gluing, bonding, or heat treating, and the like. In FIGS. 1, 2, and 5, the cover 58 is illustrated as being attached to the crotch 24, front 16, and back 18 by stitching.

Preferably, the undergarment 10 is a commercially available undergarment made of cotton and/or synthetic materials. The pad 44 may also be a commercially available product, such as a sanitary napkin or incontinence guard available at stores and pharmacies. The style of sanitary napkin or incontinence guard may vary, and the present invention may utilize absorbent pads 44 designed for both lighter and heavier fluid flows. Additionally, though the preferred embodiment of the undergarment 10 is constructed using an entire absorbent pad 44, it also may be assembled using only a portion of an absorbent pad.

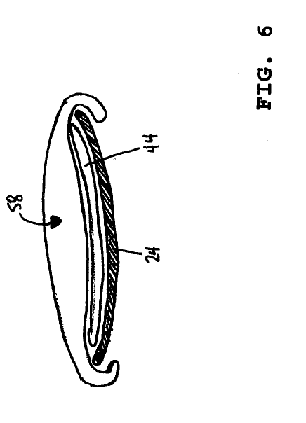

Assembly and manufacture of the undergarment 10 is easy and straightforward because commercially available “off-the-shelf” products are used. This makes manufacture and production of the invention economical and cost-effective. First, the bottom surface 43 of a standard pad 44 is placed against the inside 12 of the crotch portion 24. Next, if a cover portion 58 is to be included, it is placed over the pad. The sides of the cover 64, 66 may then be wrapped around the crotch portion's sides 54, 56, to rest against the outside 14 of the crotch portion 24. A cross-sectional view of an assembled crotch portion 24, pad 44, and optional cover 58 is shown in FIG. 6. Finally, in the illustrated embodiment, both the pad 44 and the cover 58 are stitched into place. Stitches are made along the sides 54, 56 of the crotch portion 24, through a “double thickness” of the cover 58. Alternatively, the stitching may also pass through the longitudinal sides of the pad 44. Additional stitching may further fix the front 46, 60 and back 48, 62 ends of the pad 44 and cover 58 respectively to the undergarment 10. The pad 44 is thereby prevented from shifting, which could cause fluid leakage. In another embodiment, only a single thickness of the cover is stitched or otherwise affixed to the crotch portion to fully enclose the pad within.

Because commercially available undergarments are used, the undergarment 10 looks and feels like regular underwear, allowing wearers to avoid embarrassment associated with wearing absorbent pads 44 in semi-public places, such as locker rooms, dressing rooms, and the like.

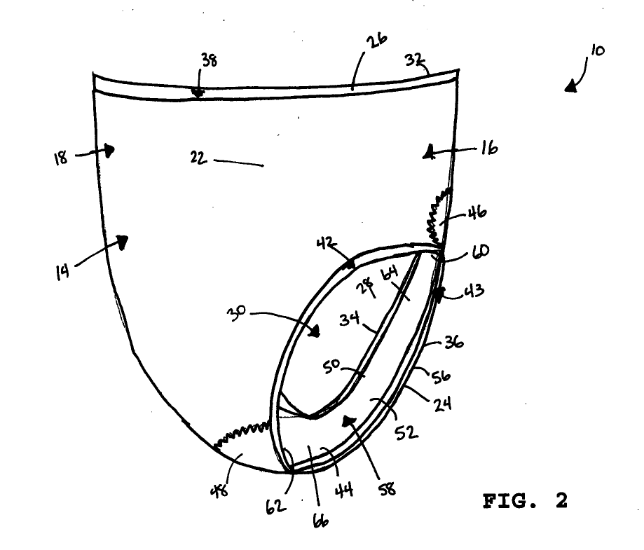

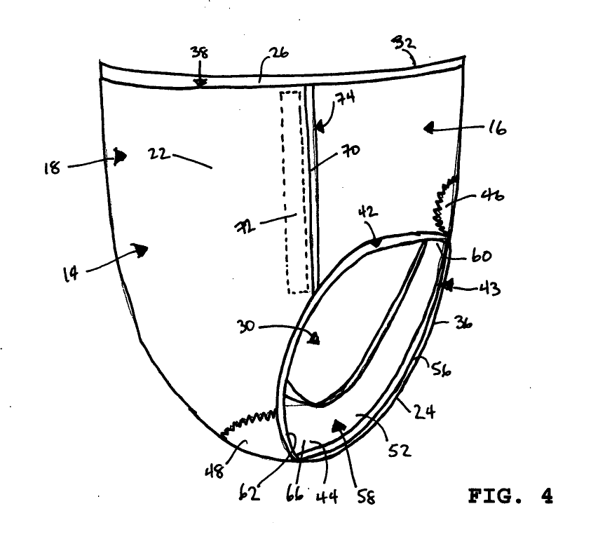

As seen in FIGS. 2, 4, and 5, an alternate embodiment is described. In this embodiment, first 68 and second 70 separation lines extend along the first 20 and second 22 side portions of the undergarment 10. The separation lines or portions 68, 70 are discontinuities or “cuts” in the material of the undergarment, and extend between the waist opening 26 and an adjacent leg opening 28, 30 respectively. The undergarment 10 is separable along both of the separation lines 68, 70 for easy removal of the undergarment from the wearer without requiring the wearer to slip his or her leg through the leg openings 28, 30. This feature is valuable for mobility-impaired individuals and the elderly, as it allows such individuals to remove the undergarment 10 with a minimum of awkward movement.

One or two separation portions may be included. If one separation portion is included, the undergarment 10 may be removed by slipping a single leg out from within the leg opening. If two separation portions are included, the entire undergarment 10 may be removed without positional change of the wearer's legs.

Preferably, complementary strips of a corresponding hook and loop fastener, such as VELCRO 72, 74 are attached to the separation portions 68, 70. To allow the undergarment 10 to close smoothly and relatively seamlessly, one strip of the hook and loop fastener 72 may be placed on the inside 12 of the separation line 68, and the complimentary strip of hook and loop fastener may be placed on the outside 14 of the separation portion 68.

While a preferred embodiment of the disposable sanitary undergarment in accordance with the present invention has been illustrated and described, it will be understood that changes and modifications may be made therein without departing from the invention in its broader aspects. Various features of the invention are defined in the following claims, and all modifications, variations, or equivalents that fall within the true spirit and scope of the basic underlying principles of the invention are intended to be encompassed in the following claims.

1. A disposable sanitary undergarment, comprising:
an inside portion and an outside portion;
a front portion and a back portion;
a pair of side portions;
a waist opening having an outer periphery;
a pair of leg openings;
a crotch portion positioned between the leg openings;
a resiliently elastic waist band extending around the outer periphery of the waist opening to facilitate securing the undergarment on a waist of a wearer;
resilient elastic leg bands circumscribing a peripheral edge of each of the leg openings to facilitate the prevention of fluid leakage; and
an absorbent pad attached to an inside portion of the crotch portion, the absorbent pad having inwardly curved opposite longitudinal sides and arcuate opposite lateral sides.

2. The undergarment of claim 1, including a permeable cover portion configured to enclose and retain the absorbent pad against the inside portion of the crotch portion.

3. The undergarment of claim 1, wherein the absorbent pad is configured to act as a sanitary napkin.

4. The undergarment of claim 1, wherein the absorbent pad is configured to act as an incontinence guard.

5. The undergarment of claim 1, wherein the absorbent pad is attached to the inside crotch portion of the undergarment by stitching.

6. The undergarment of claim 1, wherein the absorbent pad is attached to the inside crotch portion of the undergarment by chemical adhesive.

7. The undergarment of claim 1 wherein bonding means attaches the absorbent pad to an inside crotch portion of the undergarment.

8. The undergarment of claim 1 wherein the absorbent pad is attached to the inside crotch portion of the undergarment by heat bonding.

9. The undergarment of claim 2 wherein the cover portion is attached to the sides of the crotch portion by stitching means.

10. The undergarment of claim 2 wherein the cover portion is attached to the sides of the crotch portion by a chemical adhesive.

11. The undergarment of claim 2 wherein the cover portion is attached to the sides of the crotch portion by bonding means.

12. The undergarment of claim 2 wherein the cover portion is attached to the sides of the crotch portion by heat treating means.

13. The undergarment of claim 1 wherein a portion of the undergarment is discontinuous from an upper edge of the waist band to at least one of the leg openings so as to define a separation portion having first and second ends transverse to the waist band, the first and second ends being removeably separable from each other to facilitate removal of the undergarment from the wearer without requiring the wearer to slip an entire leg through the leg opening of the undergarment.

14. The undergarment of claim 13 wherein complementary strips of a cooperating hook and loop material are attached to first and second ends of the separation portion.

15. The undergarment of claim 13 further including two separation portions, each separation portion located on opposite sides of the waist band.

16. The undergarment of claim 15 wherein each separation portion includes complementary strips of the cooperating hook and loop material.

17. The undergarment of claim 1 wherein the absorbent pad is a commercially available absorbent pad.

18. A disposable sanitary undergarment, comprising:
an inside portion and an outside portion;
a front portion and a back portion;
a pair of side portions;
a waist opening having an outer periphery;
a pair of leg openings;
a crotch portion positioned between the leg openings;
a resiliently elastic waist band extending around the outer periphery of the waist opening to facilitate securing the undergarment on a waist of a wearer;
resilient elastic leg bands circumscribing a peripheral edge of each of the leg openings to facilitate the prevention of fluid leakage;
an absorbent pad attached to an inside portion of the crotch portion, the absorbent pad having inwardly curved opposite longitudinal sides and arcuate opposite lateral sides; and
a permeable cover portion configured enclose and retain the absorbent pad against the inside portion of the crotch portion, the cover portion covering the pad and inside portion of the crotch portion and wrapping around an outside portion of the crotch portion to provide a double layer of cover around the pad.

19. The undergarment of claim 17 wherein the cover portion and the pad are secured in place by stitching along opposite longitudinal edges.

20. The undergarment of claim 17 wherein the stitching penetrates the material of the cover portion, the pad and the crotch portion.
